SEQUENCE LISTING (6 sequences)

SEQ ID NO: 1 - DNA, 101 bp, Artificial Sequence
  Description of Artificial Sequence synthetic oligonucleotide
aagggccatcaggatcatcatcatgnnnacctgtattttcagggcacgtaggcgtccctgtatcaacatgccaatcaggtgcaacgtcacgatgcaaaact

SEQ ID NO: 2 - DNA, 101 bp, Artificial Sequence
  Description of Artificial Sequence synthetic oligonucleotide
aagggccatcaggatcatcatcatgagtnnntgtattttcagggcacgtaggcgtccctgtatcaacatgccaatcaggtgcaacgtcacgatgcaaaact

SEQ ID NO: 3 - DNA, 101 bp, Artificial Sequence
  Description of Artificial Sequence synthetic oligonucleotide
aagggccatcaggatcatcatcatgagtaccnnnattttcagggcacgtaggcgtccctgtatcaacatgccaatcaggtgcaacgtcacgatgcaaaact

SEQ ID NO: 4 - DNA, 100 bp, Artificial Sequence
  Description of Artificial Sequence synthetic oligonucleotide
caacgtcacgatgcaaaactaaacctggacgaatttgctagcaccatgcagtggcgctctgatggcgaagacgcgctgctggatgttggcagtggttccg

SEQ ID NO: 5 - DNA, 100 bp, Artificial Sequence
  Description of Artificial Sequence synthetic oligonucleotide
caacgtcacgatgcaaaactgatcctnnncgaatttgctagcaccatgcagtggcgctctgatggcgaagacgcgctgctggatgttggcagtggttccg

SEQ ID NO: 6 - DNA, 100 bp, Artificial Sequence
  Description of Artificial Sequence synthetic oligonucleotide
caacgtcacgatgcaaaactgatcctggacgaatttgcccgcaccatgcagtggcgctctgatggcgaagacgcgctgctggatgttggcagtggttccg

(19) United States
(12) Patent Application Publication
(10) Pub. No.: US 2014/0121118 A1
(43) Pub. Date: May 1, 2014

(54) METHODS, SYSTEMS AND COMPOSITIONS REGARDING MULTIPLEX CONSTRUCTION PROTEIN AMINO-ACID SUBSTITUTIONS AND IDENTIFICATION OF SEQUENCE-ACTIVITY RELATIONSHIPS, TO PROVIDE GENE REPLACEMENT SUCH AS WITH TAGGED MUTANT GENES, SUCH AS VIA EFFICIENT HOMOLOGOUS RECOMBINATION

(71) Applicant: OPX BIOTECHNOLOGIES, INC., BOULDER, CO (US)

(72) Inventor: Joseph R. Warner, SAN CLEMENTE, CA (US)

(21) Appl. No.: 14/080,519

(22) Filed: Nov. 14, 2013

Related U.S. Application Data
(63) Continuation of application No. 13/304,221, filed on Nov. 23, 2011.
(60) Provisional application No. 61/416,732, filed on Nov. 23, 2010.

(51) Int. Cl. C12N 15/10

(52) U.S. Cl. CPC C12N 15/1065
USPC 506/4; 435/193; 506/11; 506/38; 506/39; 536/23.2

(57) ABSTRACT

A method is provided to construct libraries of nucleic acids that comprise a plurality of mutations, each of which may be identified with particularity, such as following a selection or screening.

### CROSS-REFERENCE TO RELATED FILINGS

This application is a continuation of U.S. application Ser. No. 13/304,221, filed Nov. 23, 2011, which claims benefit to U.S. Provisional Patent Application No. 61/416,732, filed Nov. 23, 2010, all of which are incorporated by reference in their entireties.

### STATEMENT AS TO FEDERALLY SPONSORED RESEARCH

This invention was made with United States Government support under DE-AR000088 awarded by the United States Department of Energy. The United States Government has certain rights in this invention.

### BACKGROUND

Although advances have been made in the arts of genomics tools and molecular biology, there remains a need for more efficiently and clearly evaluating and identifying particular mutants that provide a desired characteristic when encoded and expressed as a polypeptide.

### SUMMARY

In various embodiments the invention comprises method to evaluate and/or identify biological nucleic acid and polypeptide constructs comprising the steps:

a. obtaining or preparing a first library of nucleic acid sequence variants;

b. obtaining or preparing a second library for which additional components are attached to members of the first library;

c. subjecting the second library to a desired protocol to enrich a subset thereof based on one or more selections or screens; and

d. identifying, sequencing, and/or quantifying one or more of the subset for its nucleic acid sequence variants.

In further embodiments this method additionally comprises attaching barcode sequences as a further additional component, thereby providing for identification of specific variants.

In any of these embodiments each of the template priming sequences may function as a respective barcode sequence.

The invention also comprises polypeptide sequence identified based on any of the methods described herein, as well as a nucleic acid sequence, such as an oligonucleotide, identified based on the methods described herein.

Also, the invention contemplates a system for identifying a selected for or screened for sequence comprising an apparatus practicing any method described herein. Further aspects of the invention may include mapping activity by mutation on the protein sequence, such as to better understand sequence-activity relationships, and transferring the mutations (variants) that performed within a set performance criterion (such as the best-performing mutations) to each member of a library, such as by standard mutagenesis, and repeating the selection method evaluating different mutation combinations.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIGS. 1-6 diagrammatically depict aspects of the present invention as it may be practiced in one or more embodiments, including in the steps provided in these figures.

FIG. 1 shows, when using a saturation mutagenesis approach, every codon is changed to every possible amino acid substitution. However, alternative mutational strategies may use this same method, such as focusing on particular regions and/or particular amino acid substitutions. A library of oligonucleotide sequences (“oligos”) is designed. Shown is an example of a design. A gene is divided up into overlapping regions. The terminal 20 base pairs (bp) of each region are conserved for amplification. In a central portion a 3 bp mutation is made that will cause an amino acid substitution. Every codon is changed to every possible amino acid for that position. The sequences to the left, spelling “Protein”, may be considered as Protein Coding Sub-Sequences and abbreviated as “PCSSx” elsewhere herein, where x may be an integer such as to designate a region for which mutants are constructed.

FIG. 2 diagrammatically depicts further aspects of the invention. Appended to the downstream side of each oligo described for FIG. 1 are cut site (CS), template priming site (TP), and unique barcode sequence and barcode priming site (Tag). In some embodiments, all oligos may be purchased as a pool, or synthesized/cleaved from a microarray (such as provided by Agilent). The cut site, CS, may be any sequence amenable to controlled cleavage, such as an endonuclease target site, Uracil DNA base, etc.

FIG. 3 diagrammatically depicts further aspects of the invention. Separately (or in multiplex, not shown) PCR amplify oligos into pools binned by gene region, remove one strand by common methods to create ssDNA (example, Lambda exo).

FIG. 4 diagrammatically depicts further aspects of the invention. Use an oligo pool as primer with another primer complementary to plasmid template and barcode priming site. PCR amplify wild-type protein coding sequence containing part of a plasmid as template. This may be done in parallel for each oligo pool or in multiplex (only one oligo pool shown from here on). 3′ end of the primer that is used is complementary to the plasmid template, and 5′ end of the primer is complementary to the 5′ end of the oligo library, therefore, double stranded circles are created, with both strands nicked. (Black lines denote connectivity.)

FIG. 5 diagrammatically depicts further aspects of the invention. Ligate circular products. Cut circular products between gene mutation region and the template priming region. Cut site is designed to allow formation of ssDNA end(s). Cut site nucleotides may be completely removed.

FIG. 6 diagrammatically depicts further aspects of the invention. Add to each pool a template DNA sequence containing wild-type (WT) Protein coding sequence and remainder of plasmid sequence. The linear mutant library can be filled in and recircularized by several means i) if mutant library has a 3′ ssDNA overhang, DNA polymerase fills in the remainder of the bases using template followed by ligation, or ii) template may be used to fill in both strands and generate a nicked circular product in a PCR reaction (shown). Another alternative is to add a unique DNA fragment to each pool of mutants that has complementary ends allowing ligation and circularization. The barcode tagged mutant library is now ready for transformation and activity assessment. Each particular set of PCSSx (depicted in the figure as “Prot”) and Tag comprise a unique construct (the latter allowing for identification) and is among a plurality of constructs for one particular region of the mutated protein coding sequence. The entire library (which may be the pooled libraries of all regions) may be assessed by selection or screening.

### DETAILED DESCRIPTION OF EMBODIMENTS OF THE INVENTION

Embodiments of the present invention are directed to construction and use of multiple sequences, such as libraries of sequences, such as nucleic acid sequence libraries, that are useful to assess which of the sequences so constructed afford particular benefit, such as increased and/or more specific and/or more particular activity by a polypeptide encoded by any one or more of the nucleic acid sequences that may be component(s) of the sequences taught herein.

In some embodiments, one may construct every possible single amino-acid change in a protein at a low cost and rapidly identify, compare, rank, and/or evaluate the activity of each mutant while using relatively few resources. In contrast, many current methods are laborious and allow only a subset of all mutations to be created and/or evaluated.

Other uses include but are not limited to:

1) Rapidly construct and evaluate mutations across multiple protein coding genes (homologous or heterologous).

2) Rapidly construct and evaluate mutations of specific regions of one or multiple protein coding genes.

These mutant genes need not reside on the vector in which library construction took place. Chromosomal gene(s) (or gene(s) on a destination plasmid) may be replaced with tagged mutant genes such as by (but not limited to) lambda RED catalyzed homologous recombination. This may be accomplished by two general methods.

1) Linear DNA containing chromosome homology or destination plasmid homology (at least about 50 bp homology required for efficient homologous recombination) flanking an antibiotic resistance cassette, barcode tag, and gene mutant is created by i) PCR amplification of gene mutant library with primers that contain homology regions, or by ii) restriction endonuclease digestion of plasmid-based gene mutant library that has destination homology already included.

Note: If gene replacement by efficient homologous recombination is the final goal, then the barcoded gene mutant library may be constructed in an analogous way as the plasmid-based mutant library, with the exceptions that the DNA mutant library need not contain a plasmid origin of replication and the DNA mutant library need not be circular.

2) The tagged mutant gene is present within a cell on a “donor” plasmid that contains two regions of chromosome homology or destination plasmid homology (at least about 50 bp homology required for efficient homologous recombination) flanking an antibiotic resistance cassette, barcode tag, and gene mutant. Within the same cell are one or more lambda RED genes (or other genes required for efficient homologous recombination) present on the chromosome, destination plasmid, or donor plasmid. Efficient homologous recombination is allowed to take place creating a replacement of wild-type gene with an antibiotic resistance cassette, barcode tag and mutant gene. Recombinants are selected under conditions where donor plasmid will not replicate (examples: temperature sensitive origin of replication, or donor plasmid containing homing endonuclease sites in a cell conditionally expressing homing endonuclease (see Gene Gorging: C. Herring, Gene 331 (2003) 153-163).

As is understood by those skilled in the art, by barcode or barcode tag is meant a unique sequence, such as but not limited to about 20 base pairs in length, which provides for identification of the construct to which it is attached. Identification may be made by sequencing, use of an array, or any other method known to those skilled in the art.

Also, it is understood that all of the sequences provided in the figures are meant for example and demonstration only, and do not indicate any particular sequence necessitating a sequence listing.

Also, as to the subscript references in the figures for the Tag, these ranges are meant to illustrate that various portions or regions of a protein sequence may be mutated by mutation of the respective codons, and each of these mutant sequences may be associated with a particular or unique tag (e.g., barcode tag). For example PCSS1 may comprise 500 mutants over a first portion of an encoded protein, corresponding to variant nucleic acid sequences in the constructs depicted in the figures, and PCSS2 may comprise 500 mutants over a second portion of the encoded protein, and so forth. In FIGS. 2 and 3 such protein coding sub-sequences are represented respectively by “PROT,” “TEIN,” and “CODI.”

A description of a non-limiting general method of the present invention, as exemplified with a specific protein, is summarized as follows:

1) Construct library of single amino acid mutants of JHAMT (juvenile hormone acid methyl transferase) protein using oligonucleotide libraries. Library size is 19 amino acids possible at every point X 297 amino acids in length=5643 mutants. Each mutant is on a plasmid. Coupled to each mutant is a 20 bp unique identifier (barcode tag) that can be used to identify and quantitate each member in the population. Exemplary construction of the mutant library (first library) and the plasmid construct library (second library) is described herein including in the appended Figures which are part of this application.

2) Sort protein mutants by fluorescence-activated cell sorting (FACS) based upon levels of fatty-acid methyl transferase activity.

3) Identify the effect of each (typically of a subset, such as those sorted in step 2 above) amino-acid change on fatty-acid methyl transferase activity using barcode technology, such as using a specific microarray.

The following summarizes aspects of the predicted quality of results:

1) An error rate in DNA sequence of about 10% is anticipated. These errors arise from the oligonucleotide library and are usually deletion of a base (The error rate may be reduced by using shorter oligonucleotides, see “note” below). Errors in sequence may lead to 3 effects:

A) The vast majority of these errors that occur within the JHAMT gene will lead to a frameshift mutation in the JHAMT protein, causing an inactive protein. These will be reported after selection and tag analysis as false negatives. For this reason (and others) proteins showing a decrease in activity should not be analyzed. These errors that occur near the C-terminus of the protein may lead to mutant proteins with improved activity that cannot be accurately identified by barcode tags. Due to this, it may be desirable to exclude the C-terminus from saturation mutation. An alternative is to use oligos with a lower error rate for mutating the downstream region of the protein coding gene (accomplished using shorter oligonucleotides or purified oligonucleotides).

B) Errors that occur within a Tag region (or Tag amplification sites) may lead to reduced detection of that Tag, if a correct copy of that tag is present within the population this error will have little/no effect.

C) The error results in misidentification of the mutant protein and therefore may result in incorrect evaluation of a particular mutant protein.

2) Effects of mutation on protein activity may be interpreted as those affecting catalysis or as those affecting protein concentration.

3) Ranking of each mutation as to its effect on activity is likely improved by increasing the number of activity “bins” that cells are sorted into.

4) The frequency of each barcode tag after FACS should be compared to the frequency of that barcode tag prior to selection such that differences in tag hybridization/detection are normalized.

Further, a brief description of procedures of embodiments of the present invention follows. These, and the particular gene/protein in the discussion, are not meant to be limiting.

Two general cloning routes are envisioned for generation of these mutant libraries, ligation based or PCR based, or a combination thereof. Described here is the PCR based route. Also, an efficient homologous recombination approach may be employed for integration.

Note: The design can be adjusted to reduce labor (fewer regions subdivided by using longer oligos, or PCR reactions carried out in multiplex. The design can be adjusted to reduce error rate by using shorter oligonucleotides and more regions. This comes at the potential cost of increased labor.

1) Design of oligonucleotide libraries: Design 17 100-base oligonucleotides that divide the JHAMT gene into 17 regions and overlap by about 40 bp. These oligos are homologous to the JHAMT gene, except for a mutation of 3 consecutive bases within the central 60 bases of each oligo that will create each particular amino-acid substitution. The terminal 20 bases of each oligo are used for PCR priming the wild-type JHAMT template. Append to the downstream side of each oligo (in this order): a cut site, such as a restriction endonuclease site, a template priming site, in this case complementary to JHAMT plasmid template, a unique Tag sequence, and a Tag (barcode) amplification priming site (see figure). In this example the total length of each oligonucleotide is approximately 165 bases long and approximately 5700 sequences are required to create every amino acid substitution. Also several barcoded wild-type JHAMT genes may be made for use as internal standards.

2) Cloning: PCR amplify using as template the pooled oligonucleotides library in 17 reactions using 17 different upstream primers such that the products are binned by the region of the gene they will mutate (binning may not be required but avoids the complications of multiplex PCR). Generate ssDNA from the PCR products by standard methods.

3) PCR amplify using: the oligo library as primer, a primer complementary to plasmid template, a JHAMT template containing JHAMT gene and part of plasmid (see attached figure for further description). Ligate PCR products (intramolecular) into circles. Nuclease treat circles, cutting between the Tag and JHAMT fragment generating linear DNA.

At this point every linear DNA fragment contains its unique mutation and tag, and is flanked by common regions complementary to the plasmid and JHAMT gene. PCR amplify (or polymerase “fill in”) each linear DNA segment using as template the other part of the wild-type plasmid containing the antibiotic resistance cassette and JHAMT gene. Products may be ligated (intramolecular) then transformed into E. coli, or products may be directly transformed.

4) Identify mutants by common Tag4 array protocols, verify a subset by sequencing.

5) Carryout selections or screens, linking activity of each cell to each barcode tag.

6) Map activity by mutation on protein sequence.

If desired, transfer best mutations to each member of the library by standard mutagenesis protocols repeat selection and barcode tag analysis.

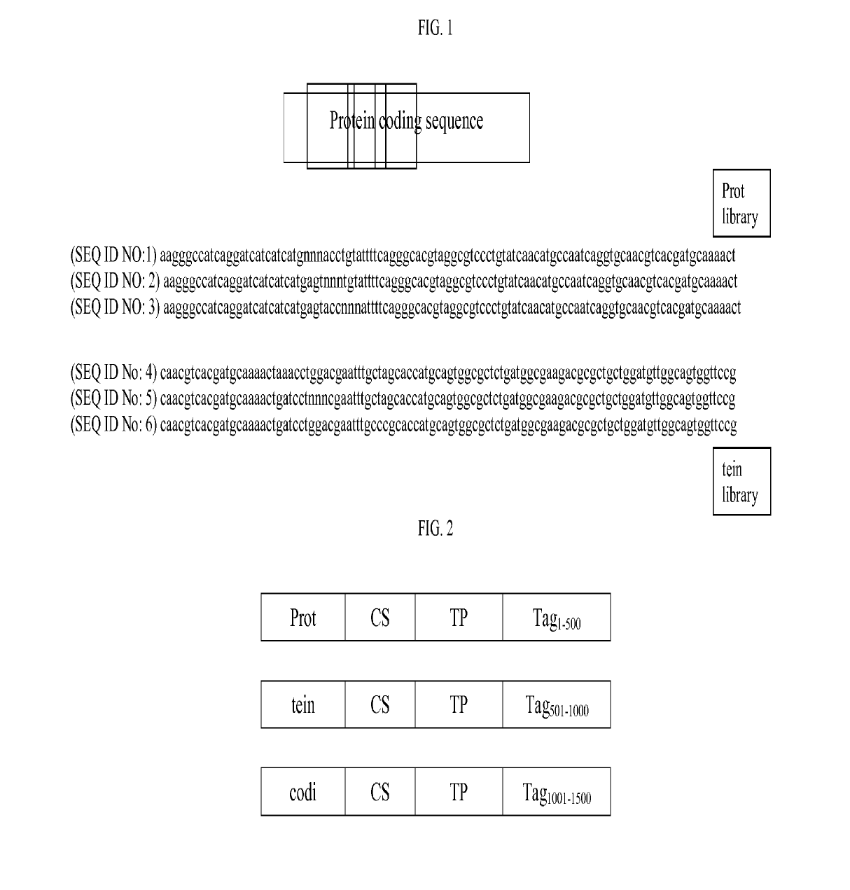

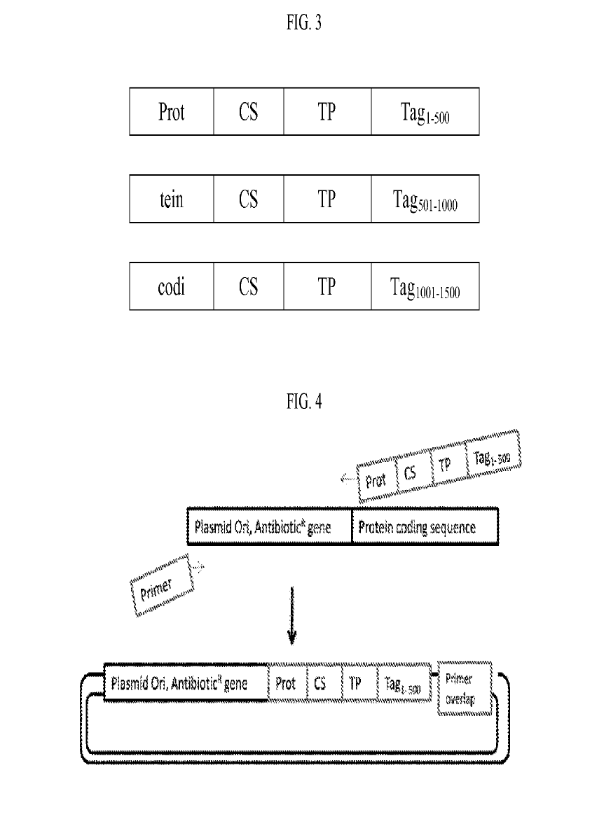

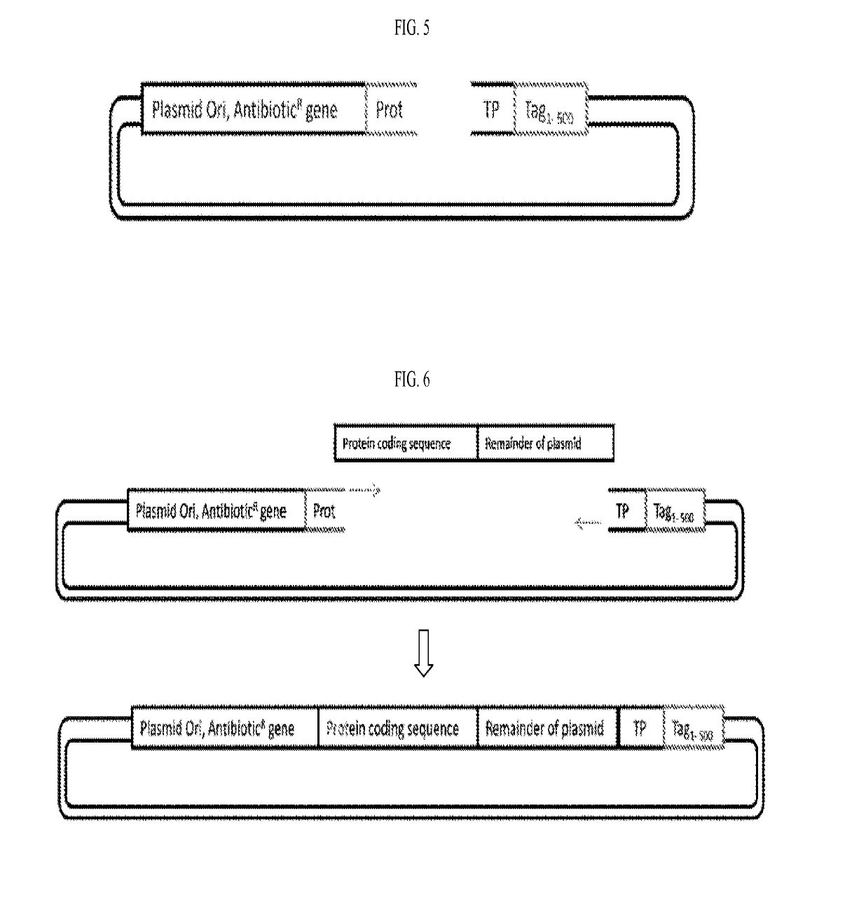

The appended FIGS. 1-6 are provided to further describe this approach. Sections of the sequence that comprises respective mutations are referred to therein as PCSSx, where x is 1, 2 or 3, to clarify that there are groups of various smaller portions of the sequence (nucleic acid corresponding to encoded protein), each group (PCSS1, PCSS2, PCSS3, etc.) corresponding to a collection of mutants along a region or portion of the protein. PCSS is intended to mean “Protein Coding Sub-Sequence” recognizing that the sequence referred to in the figures is a nucleic acid sequence.

Additional approaches and designs include:

a) Rather than target a complete protein coding gene, the oligonucleotide libraries may be designed to target only a portion of a gene and/or target multiple genes.

b) The length of each region of a particular oligonucleotide (as examples, PCR priming regions, genetic region containing mutation, etc.) may be adjusted to any length provided that each region can still perform its function and the total length of the oligonucleotide does not surpass a length that can be synthesized.

c) The template priming site “TP” could be substituted with any DNA sequence that would allow subsequent ligation of DNA ends (as examples, restriction endonuclease site, 5′ phosphorylated DNA end).

d) The template priming site “TP” may be part of or also used as a barcode tag priming site.

Additionally, it is appreciated that the PCR primers used for the PCR amplification do not need to have regions that are 5′ complementary to the oligonucleotide library. This region which creates overlap of DNA sequence at the ends (shown as “primer overlap”) is not required. The ends of the DNA sequence after PCR is carried out may be connected by another method such as 5′ phosphorylation followed by blunt end ligation.

Various genetic manipulations are conducted by means known to those skilled in the art, for instance methods taught in Sambrook and Russell, Molecular Cloning: A Laboratory Manual, Third Edition 2001 (volumes 1-3), Cold Spring Harbor Laboratory Press, Cold Spring Harbor, N.Y. See also http://chemogenomics.stanford.edu/supplements/04tag/ as to barcode tagging technology, and use of microarrays for detection.

While various embodiments of the present invention have been shown and described herein, it is emphasized that such embodiments are provided by way of example only. Numerous variations, changes and substitutions may be made without departing from the invention herein in its various embodiments. Specifically, and for whatever reason, for any grouping of compounds, nucleic acid sequences, polypeptides including specific proteins including functional enzymes, metabolic pathway enzymes or intermediates, elements, or other compositions, metabolic (including biosynthetic) pathways or portions thereof, or concentrations stated or otherwise presented herein in a list, table, or other grouping (such as metabolic pathway enzymes shown in a figure), unless clearly stated otherwise, it is intended that each such grouping provides the basis for and serves to identify various subset embodiments, the subset embodiments in their broadest scope comprising every subset of such grouping by exclusion of one or more members (or subsets) of the respective stated grouping. Moreover, when any range is described herein, unless clearly stated otherwise, that range includes all values therein and all sub-ranges therein.

### EXAMPLES

The following example is provided as one non-limiting prophetic example.

### Example 1

### Description of General Method Exemplified With a Specific Protein

Construct library of single amino acid mutants of JHAMT (juvenile hormone acid methyl transferase) protein using oligonucleotide libraries. Library size is 19 amino acids possible at every point X 297 amino acids in length=5643 mutants. Each mutant is on a plasmid. Coupled to each mutant is a 20 bp unique identifier (barcode tag) that can be used to identify and quantitate each member in the population. Construction proceeds in accordance with FIGS. 1-6.

Cells comprising these constructs that encode respective protein mutants are sorted by FACS based upon levels of fatty-acid methyl transferase activity. Thereafter the effect of each (or one or more) amino-acid changes on fatty-acid methyl transferase activity is identified using barcode technology, such as using a specific microarray.

What is claimed is:

1. A method to evaluate and/or identify biological nucleic acid and polypeptide constructs comprising:
(a) obtaining or preparing a first library of nucleic acid sequence variants;
(b) obtaining or preparing a second library for which additional components are attached to members of the first library;
(c) subjecting a second library to a desired protocol to enrich a subset thereof based on one or more selections or screens; and
(d) identifying, sequencing, and/or quantifying one or more of the subset for its nucleic acid sequence variants.

2. The method of claim 1, additionally comprising attaching barcode sequences as a further additional component, thereby providing for identification of specific variants.

3. The method of claim 1, wherein the template priming sequences function also as the barcode sequences.

4. The method of claim 2, wherein the template priming sequences function also as the barcode sequences.

5. A polypeptide sequence or a nucleic acid sequence identified based on the method of claim 1.

6. A polypeptide sequence or a nucleic acid sequence identified based on the method of claim 2.

7. A polypeptide sequence or a nucleic acid sequence identified based on the method of claim 3.

8. A polypeptide sequence or a nucleic acid sequence identified based on the method of claim 4.

9. A system for identifying a selected for or screened for sequence comprising an apparatus practicing the method of claim 1.

10. A system for identifying a selected for or screened for sequence comprising an apparatus practicing the method of claim 2.

11. A system for identifying a selected for or screened for sequence comprising an apparatus practicing the method of claim 3.

12. A system for identifying a selected for or screened for sequence comprising an apparatus practicing the method of claim 4.
